(19) United States
(12) Patent Application Publication
(10) Pub. No.: US 2008/0262626 A1
(43) Pub. Date: Oct. 23, 2008

(54) Femoral sleeve for hip resurfacing

(75) Inventor: Patrick Raugel, Ramsey, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/788,050

(22) Filed: Apr. 18, 2007

(51) Int. Cl. A61F 2/42

(52) U.S. Cl. 623/22.15

(57) ABSTRACT

A hip resurfacing femoral prosthesis has a sleeve component with an internal bore adapted to receive a femoral head and a partially conical outer surface. The sleeve is for use with a mating partial ball component shaped to conform to an acetabular socket. The sleeve is slotted or segmented to enhance the engagement with the femoral head. The partial ball component may be translated proximally and distally to reposition the outer surface by selecting sleeves with varying geometries.

### BACKGROUND OF THE INVENTION

The present invention relates generally to systems, kits and methods for joint replacement using multiple components. More specifically, in one embodiment, the present invention includes as components a ball component and an improved sleeve component for adapting the ball component to a prepared femoral head.

Artificial joint prostheses are widely used today, restoring joint mobility to patients affected by a variety of conditions, including degeneration of the joint and bone structure. Typically, the failed bone structure is, after surgical preparation of the sound bone, replaced with an orthopedic implant that mimics, as closely as possible, the structure of the natural bone and also performs its functions. The satisfactory performance of these implants can be affected not only by the design of the component itself, but also by the surgical positioning of the implanted component and the long-term fixation of the implant. Improper placement or positioning of the implant can adversely affect the goal of satisfactorily restoring the clinical bio-mechanics of the joint, as well as impair the adequate fixation of the implant to the implant to the bone.

Orthopedic implants are constructed from materials that are stable in biological environments and withstand physical stress with minimal or controlled deformation. Such materials must possess strength, resistance to corrosion, biocompatibility, and good wear properties. Also, the implants include various interacting parts, which undergo repeated long-term physical stress inside the body.

For these reasons, among others, the bone/implant interface and the connection between various parts of the implant must be durable and resistant to breakdown. This is especially important because revision of an installed implant, and the installation of a replacement implant, can be difficult and traumatic.

The requirements for the useful life of the implant continue to grow with the increase in human life expectancy. Also, as implants improve in function and expected longevity, younger patients are considered as implant candidates. It is therefore desirable to develop implants that, while durable in their own right, minimize the difficulty of revision surgery should the implant eventually fail.

There are various methods of establishing the bone/implant interface. For example, a hip joint includes ball-in-socket structure. The structure includes a rounded femoral head and a cup-like socket (acetabular cup) in the pelvis. The surfaces of the natural femoral head and the acetabular cup continually abrade each other as a person walks. The abrasion, along with normal loading, creates stress on the hip joint and adjacent bones. If the femoral head or the acetabular cup is replaced with an implant, this stress must be well tolerated at the bone/implant interface and by the implant's bearing surfaces to prevent implant failure.

Conventional total hip replacement implants use an intramedullary stem as part of the femoral prosthesis. The stem passes into the marrow cavity of the femoral shaft. These stem type prostheses are very successful but when they fail the stem can create considerable damage inside the bone. The implant can move about inside the bone causing the intramedullary cavity to be damaged. Because a stiff stem transmits the forces more directly into the femoral shaft, such implants have the further disadvantage that they can weaken the surrounding bone proximal to the hip joint due to stress shielding.

Early designs of femoral prostheses for artificial hips relied primarily on cemented fixation. These cements, such as polymethylmethacrylate, were used to anchor the component within the medullary canal by acting as a grouting agent between the component and the endosteal (inner) surface of the bone. While this method of fixation by cement provides immediate fixation and resistance to the forces encountered, and allows the surgeon to effectively position the device before the cement sets, it may, over time, become loose due to failure at the cement/bone or cement/stem interface. Alternative approaches to address the issue of cement failure include both biological ingrowth and press-fit type stems.

Prostheses stems designed for biological ingrowth typically rely on the bone itself to grow into a specially prepared surface of the component, resulting in firmly anchoring the stem of the implant within the medullary canal. A shortfall of this approach is that, in contrast to components that utilize cement fixation, surfaces designed for biological ingrowth do not provide for immediate fixation because it takes time for the bone to grow into the specially prepared textured features of the surface. Press-fit stems precisely engineered to fit within a surgically prepared medullary canal may or may not have biological ingrowth surfaces and typically rely on an interference fit of some portion of the component within the medullary canal of the bone to achieve stable fixation.

Press fitting a portion of an implant component having a textured ingrowth surface presents the problem that the very high friction coefficient of the rough ingrowth surface may require high forces to overcome the shear force developed between the ingrowth surface and the bone surface to seat the implant. This friction may even prevent proper seating in the desired position or prevent compression of the bone to create a sufficient press fit force to achieve fixation.

The need often arises to replace at least a portion of a hip implant. Prior art designs often require the entire implant to be replaced even if only a portion of the implant fails. Similarly, the entire implant may have to be replaced if the implant is intact but certain conditions surrounding the implant have changed. This is often due to the implant suffering from a decrease in support from the adjacent bone from stress shielding or other negative effects of the implant on surrounding bone.

Surgeons have sought a more conservative device than an implant using an intramedullary stem as part of the femoral prosthesis. There have been a number of attempts going back over fifty years at implants using short stems or femoral caps without stems and requiring less extensive surgery. Current approaches to femoral head resurfacing typically use a stem an example being the Birmingham Hip Resurfacing implant developed by McMinn in the United Kingdom.

A modular stemless approach to a femoral hip resurfacing is shown in U.S. Pat. No. 4,846,841 to Oh. in this approach, a frustro-conical cap or sleeve is press-fit to a prepared femoral head. A ball component is then attached to and retained by the cap using a Morse type taper fit. A similar approach is shown in U.S. Pat. No. 5,258,033 to Lawes and Ling, which shows a ball component cemented either directly to a prepared head or additionally retained by a press-fit with a frustro-conical sleeve.

Problems are encountered when attempting to press fit such frustro-conical sleeves onto the prepared femoral head. Firstly, as previously mentioned, high forces may be required to overcome the friction between the sleeve inner surface and the bone, resulting in distortion of the bone or sleeve or improper positioning of the sleeve. The friction problem is exacerbated by a high friction porous or textured surface and by the increasing normal force to the surfaces as the frustro-conical sleeve approaches the final position. For these reasons, obtaining a satisfactory initial press fit of sleeve with a high friction inner surface is difficult.

Secondly, driving the sleeve using the ball component or a tool fitting the sleeve taper, such as a driver, produces a strong machine taper press fit between the sleeve and the driver relative to the press fit between the bone and the driver. Thus, in the instance of fitting or re-fitting a ball component the driver cannot be removed from the sleeve without pulling the sleeve off the bone surface unless the driver is separable. In the instance of using the ball component itself to seat the sleeve, the mismatch in elasticity between the low modulus bone and the high modulus ball component means that the bone may not be sufficiently compressed by the inside cup sleeve surface to establish a satisfactory press fit on the bone as will be elaborated in the detailed description of the invention. Further, removal of the ball will tend to remove the sleeve because the bone/sleeve interface will break loose before the sleeve/ball interface.

All of these more modern hip resurfacing approaches require that the femoral head be prepared to provide a properly oriented, positioned and shaped bone interface for the implant by shaping the head. The outer prepared bone interface with the implant is usually symmetrical around an axis passing through the central region of the femoral neck and is typically cylindrical or conical but may be a more complex solid of revolution. The proximal portion of the prepared head can be a flat surface, tapered, domed, chamfered, or any combination of these features and is usually performed as a separate resection following preparation of the outer interface surface. If a stem is used, it is typically short compared to a conventional intramedullary stem. The portion of the bone that hosts the prosthesis must be shaped so that it matches the shape of the prosthesis. The size and shape of the bone may fit exactly the shape and size of the prosthesis or may provide room for cementing to take place or have an excess of bone in a region to allow press-fit fixation, depending on the preferred fixation method.

Because the desired bone shape of the outer implant interface is symmetrical around an axis, a guide wire introduced into the femoral head is typically used to establish the tooling landmark for the various measuring and cutting tools used in the preparation process by providing an axis of revolution. Based on pre-operative planning, the surgeon initially places the guide wire, either freehand or using measurement and guidance tools based on various anatomical reference points on the femur. In order to place the pin, the pin is driven or inserted in the proximal surface of the femoral head directed toward the greater trochanter and approximately down the mid-lateral axis of the femoral neck. A gauge having an extended stylus that allows measurement of the position of the pin with respect to the neck is then typically used to make a preliminary check of the pin position. By revolving the gauge, the surgeon can evaluate the position of the pin to ensure that the femoral neck will not be undercut when the cutting tool is revolved around the pin. The surgeon also uses the gauge to evaluate the support the prepared femoral head will provide to the implant and the head/neck diameter ratio. If the surgeon is satisfied that the pin position meets these criteria, the guide wire is used to establish the axis of revolution for the shaping cutter or reamer to be advanced along the pin to prepare the head. If a stem cavity is required, a cannulated drill or reamer is centered on the guide pin to create the cavity after creating the outer surface of the prepared head.

The head diameter/neck diameter ratio mentioned above is a metric wherein a low ratio indicates a risk for undercutting the neck. It is helpful in the instance of a low head diameter/neck diameter ratio if the required external preparation profile of the head for a given prosthesis is as large as possible relative to the ball component diameter.

Therefore, there is a need for a femoral resurfacing prosthesis that provides a more successful surface replacement of the femoral portion of a total hip replacement by improvements to a stemless, modular approach to femoral hip resurfacing.

### SUMMARY OF THE INVENTION

According to an aspect of the present invention, a total hip replacement femoral prosthesis has an outer ball component sized to conform to an acetabular socket and an inner sleeve component adapted to be positioned over a prepared femoral head. The ball component is hemispherical and has an internal bore adapted to receive the outer surface of a sleeve. The bore and sleeve outer surface have mating surfaces typically in the shape of a truncated cone to create a machine taper type fit, but may also incorporate anti-rotational or indexing features such as a tapered spline, tapered square or a keyway and key. The inner surface of the sleeve is shaped and dimensioned to substantially conform to a prepared femoral head. The sleeve and prepared head may also incorporate anti-rotational or indexing features. The sleeve receives the head and is retained by various known methods including bone ingrowth or an interference fit.

It is another aspect of the invention to provide sleeve components with adjustable resiliency, stiffness and deflection in order to minimize installation difficulty and maximize retention of the sleeve on the prepared head.

It is another aspect of the invention to provide the adjustable resiliency, stiffness and deflection of the sleeve components by creating gaps that separate the sleeve into segments or regions capable of individual radial deflection.

It is another aspect of the invention to provide gap geometries that increase the stiffness of the sleeve when the gap closes as a result of either a maximum or minimum radial deflection of the sleeve.

It is another aspect of the invention to provide sleeve components with a stiffness gradient or zones whereby the portion of the sleeve corresponding to the proximal portion of the head is stiffer than the portion of the sleeve corresponding to the distal portion of the head in order to match the gradient of stiffness in the trabeculae of the natural femoral head.

It is another aspect of the invention to provide sleeve components with altered geometries to allow variation of the medial-lateral location of the ball component with respect to the axis defined by the femoral head and neck.

It is another aspect of the invention to provide sleeve components with altered geometries to allow the surgeon to adjust for variation in the head/neck ratio of various patients.

In a preferred embodiment the internal bore of the sleeve component is inwardly tapered. Thus, the taper can be co-axial with the femoral neck although there may be advantages in orienting the axis of the taper slightly more vertical when in position so that it is closer to the average force vector acting on the femoral head during human activity. With this tapered sleeve the interface between the sleeve and the prepared bone is placed in compression, once the ball is installed on the sleeve, to aid in retention and facilitate bone ingrowth. The sleeve bore may be arranged with anti-rotation features such as ridges which extend along the length of the sleeve to engage the prepared bone surface and prevent rotation of the sleeve relative to the bone.

It is also an aspect of the invention to provide a kit of ball and sleeve components with not only the usual variety of sizes of ball components etc. to fit the implant to the patient but also to provide a kit of sleeve components to facilitate adjusting the ball component location during surgery with altered geometries to facilitate variation in the location of the ball component and sleeve along the neck axis by the surgeon during surgery. Such a kit may also contain trial components, such as trial components that facilitate selection of the sleeve component to actually be fitted to the patient. It is also an aspect of the invention that the various geometries of the sleeve components are marked on a surface of sleeve that will still be visible once the ball is installed. This aspect of the invention is particularly important when the geometry of a sleeve feature will not be apparent or measurable when the component is installed.

Another object of the invention is to provide a method for installing the femoral prosthesis described above by appropriately preparing and shaping the femoral head, guiding and fitting the sleeve to a proper orientation on the prepared femoral head, and guiding and fitting the partial ball component onto the sleeve to complete the installation of the prosthesis.

### BRIEF DESCRIPTION OF THE DRAWINGS

FIG. 1 is a cross-sectional side view of the upper portion of a human femur;

FIG. 1A is a close-up view of the femoral head depicted in FIG. 1;

FIG. 1B is a top view of the femoral head depicted in FIG. 1A;

FIG. 2 is a cross-sectional side view showing a sleeve and ball component installed on a prepared femoral head;

FIG. 3 is a perspective view of a sleeve and ball component;

FIG. 4 is a cross-section view of the assembled sleeve and ball component depicted in FIG. 3;

FIG. 5 is a cross-section view showing additional details of the sleeve depicted in FIG. 4;

FIGS. 6 and 7 are cross-section views of alternate sleeve configurations;

FIG. 8 is a perspective view of an embodiment of the present invention;

FIG. 9 is a cross-section view of the sleeve of FIG. 8 and a ball;

FIG. 10 is a cross-section view of a partially assembled sleeve and ball component of FIG. 9 with the sleeve fitted on a prepared femoral head;

FIG. 11 is a perspective view of a sleeve according to an additional embodiment of the present invention;

FIGS. 11A-11C show detail views of the sleeve of FIG. 11;

FIG. 12 is a perspective view of a sleeve according to an embodiment of the present invention;

FIG. 13 is a perspective view of a sleeve according to an embodiment of the present invention;

FIG. 14 is a perspective view of a sleeve according to an embodiment of the present invention;

FIG. 15 is a plan view of a sleeve depicted in FIG. 14 in its free state;

FIG. 16 is a plan view of the sleeve depicted in FIG. 14 in an expanded position;

FIG. 17 is a plan view of the sleeve depicted in FIG. 14 in a fully-compressed position;

FIG. 18 is a perspective view of a sleeve according to an embodiment of the present invention;

FIGS. 19, 20 and 21 show variations of sleeves according to an embodiment of the present invention for varying the axial position of the ball;

FIG. 22 is a cross-section view of a sleeve according to an embodiment of the present invention having a gradient of stiffness;

FIG. 23 is a cross-section view of an assembled ball and sleeve according to FIG. 22; and

FIGS. 24, 25 and 26 are cross-sectional views of embodiments of the present invention combining embodiments of the present invention.

### DETAILED DESCRIPTION

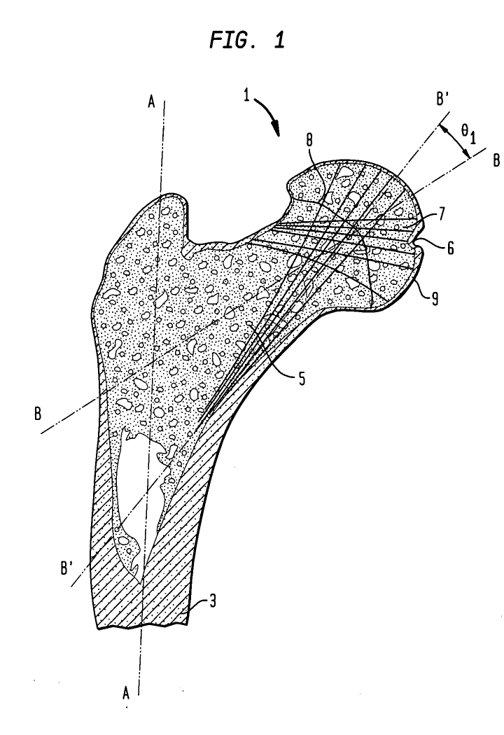

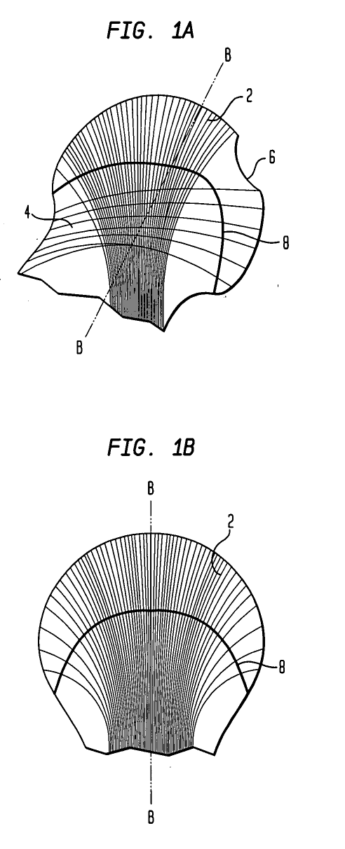

The location and function of a bone within the body typically define the mechanical properties of that bone. Bone generally comprises dense cortical bone and trabecular or cancelleous bone, which is porous and has an open cancellated structure. Considering the femoral bone of the hip joint, FIG. 1 shows the proximal portion of a femur 1 with the upper portion of the shaft 3, a neck 5 and a head 7. An axis A-A is generally aligned with the shaft 3 and an axis B-B is aligned with the neck 5. The shaft 3 is primarily composed of cortical bone while the neck 5 and head 7 are primarily composed of trabecular bone with cortical bone at the surface. FIGS. 1A and 1B indicate the main groups 2 and 4 of trabeculae in the femoral head 7 in further detail. The group 2 is the principal compressive group through which the resultant load vector at the head due to body weight and muscular force can be shown normally to pass. This group extends from the medial cortex of the femoral shaft 3 to the femoral head 7 in slightly curved lines which diverge to embrace the articular area of the head, and they are among the densest and stiffest trabeculae in the proximal femur. The group 4 is the principal tensile group and extends from the lateral cortex immediately below the greater trochanter to curve upwardly and inwardly across the neck 5 of the femur to terminate in the medially inferior portion of the head below the fovia capitis. This group is placed in tension by the moment created by the offset of the resultant load vector from the shaft axis A-A. Thus there is a gradient of stiffness in the trabeculae of the natural femoral head whereby the proximal and superior bone in the region of the resultant load vector is stiffest while the distal head region is less stiff.

As shown in FIG. 2, a proximal femur as depicted in FIG. 1 has been surgically prepared for the implantation of a femoral hip resurfacing prosthesis. The preparation consists of a re-shaping of the femoral head 7, in this instance, as a surface of revolution about the femoral neck axis B-B. The femoral head 7 has been re-shaped, by known surgical techniques, to yield a prepared femoral head 7′. The femoral head surface 9 has been removed, creating a prepared femoral head surface 9′. In accordance with the present invention, arranged in close contact with the prepared femoral head surface 9′, is a sleeve 10. In turn, a ball component 20 is fitted over the sleeve 10. In this manner, a modular prosthesis comprising the sleeve and ball is emplaced on the prepared femoral head with various embodiments and advantages as will be further shown and described.

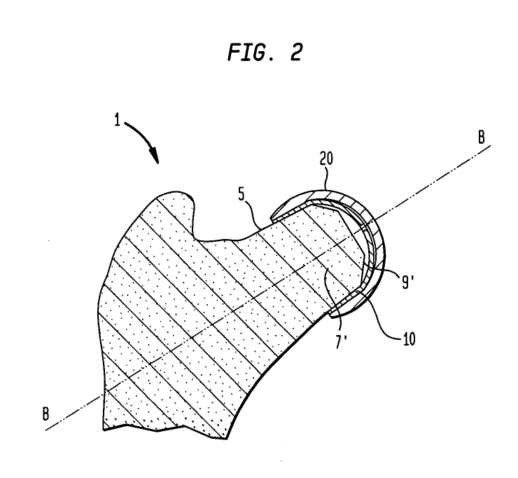

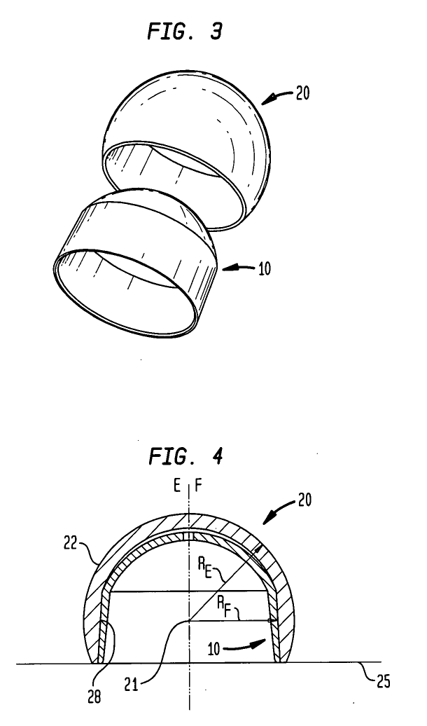

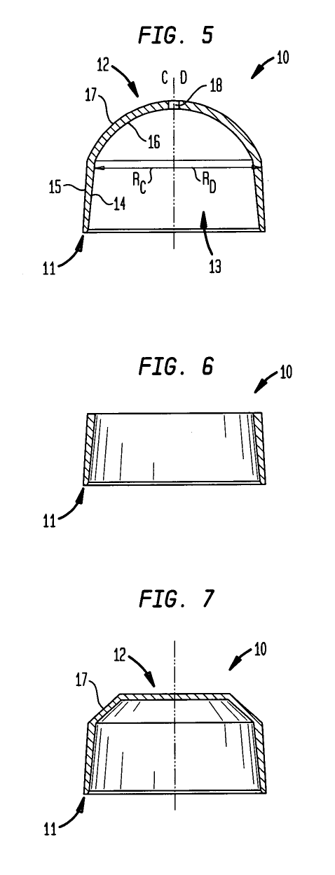

FIG. 3 shows in an exploded perspective view the prosthesis of FIG. 2. It can be seen that the sleeve component 10 in this embodiment fits closely inside at least a portion of the ball component 20. It can further be seen in FIG. 5 that the sleeve 10 is generally a shell of revolution about a central axis having a sleeve cavity 13 which is configured to interface with the prepared femoral head surface 9′.

The sleeve 10 has a distal portion 11 and a proximal portion 12. The distal portion 11 is in the configuration of a hollow truncated cone, having an inner surface 14 and an outer surface 15. Preferably, as shown in FIG. 5, the inner surface 14 and outer surface 15 are machine tapers to facilitate frictional locking on the prepared femoral head surface 9′ and the cavity of the ball component 20. Either of the machine tapers can be characterized by a cone angle θ which is typically between 3° to 12°, and preferably between 6° to 9°.

In use, the sleeve 10 is compressed by the mating taper of the interior cavity of the ball component 20 in order to generate frictional retention forces at the sleeve/ball interface. In the prior art sleeves, the deflection of the sleeve inner surface 14 caused by the compressive force applied by the mating taper is extremely small. This is because the resisting hoop stress established by the annular cross sections of the sleeve counteracts the compression. The resulting small deflection of the prior art sleeve is insufficient to substantially increase the pressure at the neck sleeve interface and aid in retention of the sleeve.

For a given position along the central axis, the inner surface 14 of the sleeve 10 can be characterized by a radius R_{c} and the outer surface can be characterized by a radius R_{d}. The sleeve inner surface 14 is a surface of revolution characterized by a radius from the central axis, R_{c}. R_{c} can characterize as a tapered or other variable surface of revolution and therefore is not to be taken as a constant radius for a given position along the axis C. For example, as shown in FIG. 5, R_{c} will be shorter in the proximal region and longer in the distal region of the distal inner surface 14 in accordance with the tapered geometry shown. In the same manner, the distal outer surface 15 of the sleeve is a surface of revolution having radii R_{d}.

The surface of revolution 14 characterized by R_{c} defines the central axis C and the surface of revolution 15 characterized by R_{d} defines a central axis D. As depicted in FIGS. 5, C and D are coincident. Thus, the axis C is defined by the sleeve inner surface 14 of the sleeve cavity 13 and is referred to here as the cavity axis. The axis D is defined by the sleeve outer surface 15 and is referred to as the sleeve axis. It noted that it is not necessary that the cavity axis C and the sleeve axis D be coincident, but for the purpose of the balance of this application, the axes will be considered to be coincident and the axis of the sleeve will be defined by axis D.

While one embodiment has a truncated cone shape with two tapering surfaces 14 and 15, either of surfaces 14 and 15 can define a hollow cylinder or other surfaces such as an ogive or any parabolic surface capable of being fit over a matched prepared femoral head surface 9′. The proximal portion 12 can be of any suitable shape of revolution about the central axis or, as shown in FIG. 6, may not even be present. When present, the proximal portion may be closely configured to the prepared femoral head surface 9′ or may have clearance from the prepared femoral head surface.

The proximal portion of the sleeve 12 has an inner surface 16 and an outer surface 17. As shown in FIG. 5, the proximal portion of the sleeve 12 can be in the configuration of a spherical dome, or alternatively, can be other suitable configurations such as the chamfered configuration shown in FIG. 7. Preferably the outer surface 15 of the distal portion of the sleeve 10 fits tightly with the matching inner surface 28 of the ball component 20. However, it can be seen, as in FIG. 4, that the proximal portion 12 is not in direct contact with the ball component 20, i.e., there is clearance with respect to the cavity of the ball component 20.

In FIG. 4, the ball component 20 is depicted in cross-section with the sleeve 10 inserted in the bore of the ball component. The hemispherical bearing surface 22 defines a center 21 having a radius Rₑ, the distal plane 25 defines the extent of the surface and also a distal surface 24. The body of the ball component 20 is preferably made of a metallic material similar to those described for the sleeve 10 with the exception that the material is typically solid throughout and has a suitable hardness and durability to provide a bearing surface or substrate. For durability and bearing performance, the ball component 20 may be coated or have a surface layer of ceramic material, or may be entirely composed of a ceramic.

A polar axis E of the ball component 20, as shown in FIG. 4, is defined by a line passing through the center 21 of the ball component 20 and perpendicular to the distal plane 25. The bore 28 is a surface of revolution defined by an axis F and radii R_{f} perpendicular to central axis F. Bore 28 can be perpendicular to the distal plane 25 and centered on the center 21 in which case axes E and F are coincident. In the examples of this specification axes E and F are coincident. However, axes E and F need not be coincident as disclosed in U.S. patent application Ser. No. 11/478,870.

It will be apparent to a person of skill in the art that when matching tapers of a Morse or other machine taper type are used for the interface of the outer surface 15 of the distal portion of the sleeve 10 with the matching inner surface 28 of the ball component 20, large compressive forces result at the interface between the sleeve and ball. This results in a correspondingly high hoop stress within the sleeve. These compressive forces decrease the inner sleeve diameter R_{c} to a certain small extent, but because of the hoop stress, the sleeve is rigid in the radial direction. Consequently, the compressive forces between the inside surface of the sleeve 14 and the surface of the prepared femoral head 9′ are substantially less than the compressive forces between the outer surface of the sleeve as will be further discussed below.

The resulting low interface force limits the initial retention force between the femoral head and sleeve. The retention force is potentially inadequate, increasing the risk of the sleeve moving relative to the bone on either a macro or micro level to create misalignment and hinder bone ingrowth. The limited interface compression and retention force also creates the situation where, for a sleeve using initial press fit retention, removal of an installed ball from a sleeve will shear the femoral head/sleeve interface and remove the sleeve along with the ball.

The sleeve 10, as depicted in FIG. 5, may be a solid structure, or it may have a porous inner surface at 14 that is integrated with or attached to a solid outer layer or the sleeve may be porous throughout. In a preferred embodiment, the sleeve has a textured or porous inner surface 14 to allow an initial retention by a press fit and later improved retention by bone ingrowth with respect to the prepared femoral head surface 9′. The sleeve may also have mechanical retention features such as spikes or ridges that impinge into surface 9′.

The structure on the inner surface of the sleeve 14 may be of a configuration to promote bone ingrowth of the prepared femoral head surface 9′ into the mating surface of the sleeve 10. The inner surface structure can be porous or textured as is known in the art. The sleeve may have gradient or zonal transitions of porosity and other pore characteristics both over the surface 14 and through the thickness of the sleeve. For example, the sleeve may be more porous at the inner surface 14 and dense at the outer surface 15.

The characteristics and fabrication of such tissue ingrowth surfaces, either porous or a textured solid, are known in the art, for example technologies such as selective laser melting can be used to create porous structures and gradient porous structures with variations of pore characteristics such as the pore size, pore interconnectivity and porosity. The porous and solid portions of the sleeve 10 are preferably made from biocompatible metals, such as titanium, titanium alloys, cobalt chrome alloy, stainless steel, tantalum and niobium. The most preferred metals are titanium and titanium alloys.

Optionally, additional bioactive materials can be incorporated in the porous sleeve inner surface 14 as are well known in the art. Examples include bone morphogenic protein to promote bone ingrowth, calcium hydroxyapatite and tricalcium-phosphate, to promote bone adhesion to the porous sleeve inner surface, and antibiotics, to reduce the potential for infections and promote healing.

Different methods may be used to transition the porosity characteristics of the sleeve 10. For example, a first region adjacent the sleeve outer surface 15 may be relatively dense, having a porosity in the range from 0% to 50% and the second porosity region adjacent to the porous inner surface 14 may have a relatively greater porosity in the range from 20% to 90%. In the instance of overlapping porosity ranges, the porosity will generally be less in the outer porosity region than in the inner porosity region. It is also possible to establish a gradient of porosity throughout the sleeve progressing from a substantially solid outer surface to a porous inner surface. The gradient of porosity through the sleeve layer may be linear, defined in zones as above or by other means. Variations in the porosity characteristics may be used to alter the modulus of elasticity of the sleeve materials and control the rigidity and transitional material properties between porosity zones, differing materials and differing structural load regions. Methods of achieving distributions of porosity are also discussed in co-owned application Ser. No. 10/317,229 entitled “Gradient Porous Implant”.

As previously discussed, the prior art sleeve designs for resurfacing implants have significant shortcomings. For a press fit application or an application requiring an initial press fit to allow bone ingrowth into a textured or porous sleeve inner surface, high friction can prevent proper positioning and the development of a sufficient press fit between the sleeve and the bone. Even more importantly, the radial rigidity of prior art sleeves prevents development of a sufficient press fit between the bone and sleeve as a result of compressing the sleeve as the ball is fitted.

An aspect of the present invention addresses these shortcomings by enhancing the ability of the sleeve to deflect radially in response to applied forces. This is accomplished by providing the sleeve with cuts that are preferably primarily aligned with the sleeve axis C to create gaps defining petal-like segments that are more or less free to deflect in the radial direction when radially loaded. As will be seen in the subsequent examples “primarily aligned” is meant here in a broad sense to indicate the trend of the cut geometry. Portions of the cut may be skew or even perpendicular to the axis to provide additional benefits as will be further elaborated. However, in all cases, the cuts will create gaps with respect to lines of circumference around the sleeve and about the central axis C that interrupt the development of a hoop stress and allow the segments defined by the gaps to flex more readily in the radial direction. Even in the instance of a single cut, regions of the segment adjacent the cut will be free to flex and provide the benefits of easier installation and greater retention force.

The cuts used to create the segmented sleeve may be created by conventional machining technologies. Wire EDM is a preferred method of creating the cuts, particularly those with complex profiles. Laser cutting is also a suitable method.

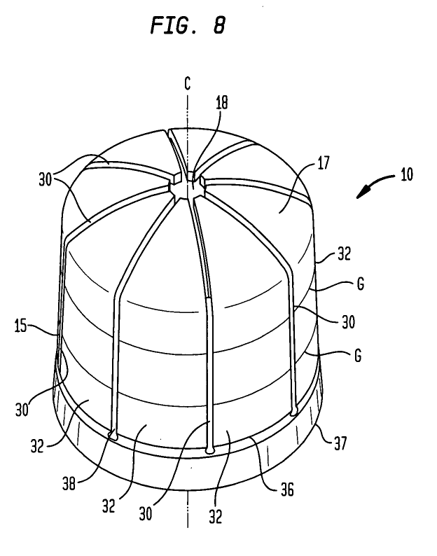

Turning to FIG. 8, a perspective view of a sleeve 10 modified by having eight cuts 30 radiating from the central axis C to create gaps. The cuts 30 divide the entire proximal portion 12 of the sleeve 10 and a substantial portion of the distal conically-tapered region of the sleeve into eight segments 32. These segments are now capable of flexing inward or outward considerably more readily than before the cuts were made. This is but a first example of a modified sleeve 10 according to the invention, and as will be shown, the number and shape of the cuts are open to considerable variation.

As previously discussed, the gaps created by the cuts 30 interrupt the development of hoop stress around the sleeve and allow the segments 32 to flex substantially independently and effectively transmit force applied to the conical outer surface 15 and the proximal surface 12 of the sleeve 10 to the prepared femoral head surface 9′. This results in an order of magnitude increase in the retention force created by installing the ball component 20 compared with the retention force created using an unmodified sleeve.

It can be seen that with the cuts 30 shown in FIG. 8, the segments 32 deflect with respect to a solid lower base 37 of the sleeve that is left uncut. The base 37 serves several functions. Firstly, while it would be possible for a sleeve 10 of the present invention to be composed of separate segments 32, with a suitable retention means, it is preferred to have a unitary structure of the sleeve 10 from both a use and fabrication viewpoint. Secondly, the un-segmented base 37 provides the advantage that the rim region 11 of the sleeve can be designed to provide a seal and prevent fluids from the joint capsule from entering the sleeve and adjacent bone under pressure as the joint articulates. Thirdly, the relatively high hoop stress established by the solid base 37 limits the distal progression of the ball 20 along the axis C to control the ball position and the compressive stress created by the ball. The fourth advantage of the solid rim is that the solid rim limits the compressive stress applied to the relatively weaker bone of the neck region 4.

Several features aid in allowing the segments 32 to flex. A central hole 18, with an axis coincident with the sleeve axis C, allows the segments 32 to flex radially inward. A relief groove 36 about the circumference of the sleeve at the distal end of the cuts 30 reduces the sleeve's thickness at the transition of each segment 32 to the base 37 to create a hinging effect and diminish the relative stiffness created by beam loading in this region. The boundary conditions at the transition can create regions within a segment that flex inward more or less readily. For example, the region at the transition will be stiffer, while an intermediate section will have a relatively larger deflection for a given load. Even a single cut 30 in a sleeve will enhance the deflection of the regions at either side of the center of the cut and allow them to move relatively independently.

The groove 36 can define a line of circumference around the sleeve 10 that falls within a plane normal to the central axis C. Additional virtual planes G of are also shown parallel to groove 36 and it can be seen that such virtual planes G will be interrupted by the cuts 30. In the example shown in FIG. 8, the cuts 30 are substantially normal to planes G at each intersection. At the distal end of each cut 30, a relief hole 38 is drilled or otherwise formed to create a stress relief. Other methods of obtaining a stress relief, as are known in the art, such as using a chamfer at the transition from the cuts 30 to the base 37 may also be employed.

While outward radial deflection of the segments 32 is essentially limited only by the forces applied and the material properties of the sleeve 10, inward deflection of the segments becomes limited when the gaps created by the cuts 30 close and the opposing segments 32 come in contact. The closed segments resist inward deflection because hoop stress is developed and now resists the inward deflection.

When the segments 32 are subject to an inward radial loading, as will be the case when the inner surface 28 of a ball 20 is mated with the distal conical outer surface 15 of the sleeve, all of the gaps 30 will close as they are compressed inward, and the sleeve structure will greatly increase in radial rigidity as hoop stress develops between the segments 32 in the same manner as a solid sleeve. Careful inspection of FIG. 8 will show that, in the free state the taper angle defined by the distal portion 15 of the segments 32 is somewhat smaller than the tapered angle defined by the base 37 so that, when deflected, the taper angle is more or less the same for the segments 30 and the base due to the hinging at the connection between the segments 32 and the base 37.

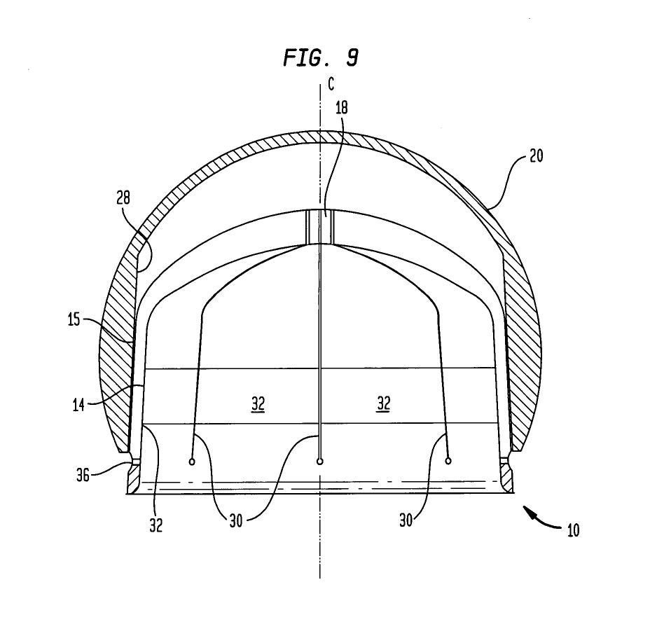

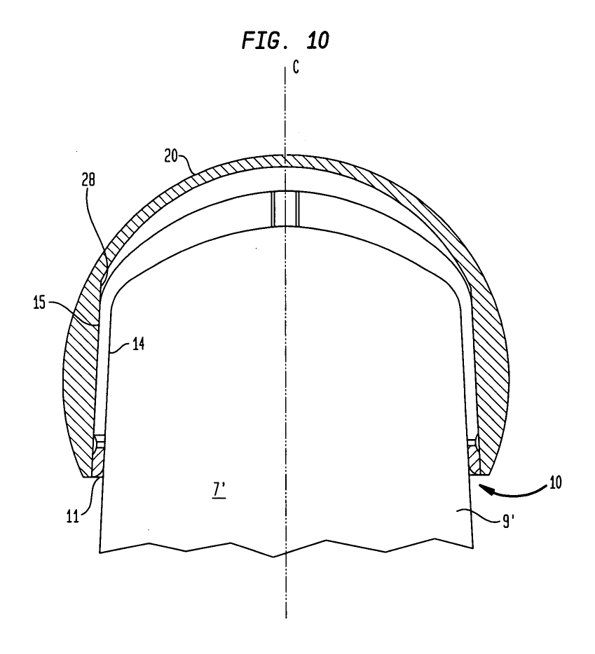

FIG. 9 shows that because of the smaller taper angle of the segments 32 in this embodiment, when a circle ball 20 is positioned over the sleeve 10, the tapered inner surface 28 of the ball first engages the more proximal conical surface 15 of the segments 32 and drives the segments radially inward. Thus, as seen in FIG. 10, the inner surface of the sleeve 14, composed of the various segments 32, is driven radially inward to create a compression fitting on the prepared femoral head surface 9′. Because the segments 32 are free to deflect radially inward, the outer surface 15 is progressively drawn inward as the ball 20 is seated on the head to create a relatively high compressive force on the surface 9′ of the prepared femoral head 7′ to aid in the initial retention of the sleeve and ball with respect to the head. Only during the final incremental travel of the ball 20 does the inner surface of the ball 28 engage the solid base 37 of the sleeve to create a higher locking force due to the hoop stress in the base section during this increment of travel on the ball as it is seated in its final position on the sleeve 10. The gaps can be sized so that they only completely close during this final increment of travel immediately after the position shown in FIG. 9. After closure, hoop stress may also be developed in the segments 32 and thus the locking force between the tapered inner surface 28 of the ball 20 and the tapered outer surface 15 of the distal portion of the sleeve 10 may be optimized to create a higher and controllable locking force during this last increment of the ball seating motion. By controlling the relative tolerancing between the surface of the prepared femoral head 9′ and the final more deflected position of the segments 32, the compressive interface stress between the bone and sleeve can be controlled for optimum retention and bone vitality. It is also possible to limit the compressive stress at the bone sleeve interface, by designing the width of the gaps created by the cuts 30 to close at a desired deflection in which case further deflection of the sleeve will be limited by the increase in radial rigidity from the sleeve 10.

A segmented sleeve 10 constructed according to an embodiment of the invention as shown in FIGS. 8, 9 and 10 provides many advantages both in function and in installation. During installation of the sleeve, the sleeve is free to expand to decrease the installation force and insure that the sleeve is fully seated on the prepared femoral head. During initial fitting of the ball on the sleeve, the flexible segments 32 are free to deform inward and compress the prepared femoral head surface 9′. The force required during the initial travel of the head onto the sleeve to a position such as shown in FIG. 9 is substantially less than in the case of an un-segmented sleeve. However, because the segments can apply suitable pressure to the bone of the prepared femoral head, the head is compressed in a controlled manner and the compressive force at the bone sleeve interface is greatly increased.

It has been found that because of the relatively high friction created between the textured or porous inner surface of the sleeve 14 and the prepared femoral head surface 9′ combined with the increased interface force, the sleeve will remain on the head should the ball need to be later removed.

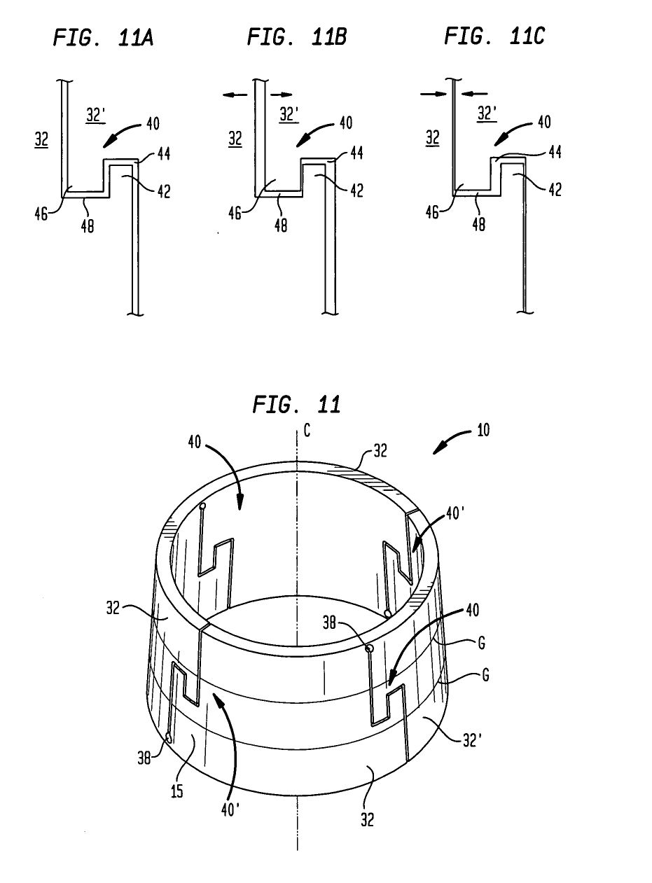

FIGS. 11 through 18 show additional variations of this aspect of the invention using slots primarily aligned with the sleeve's central axis C to allow controlled radial flexing of the sleeve's segments and create the benefits described above. The various geometries of slots shown are but examples and a great many options are available to allow the implant designer to alter the flexibility of a sleeve and create a desired result. For example, all of the slots shown are symmetrically reflected on the opposite side of the sleeve. It may be desirable to create more enhanced radial deflection in a particular area by adding additional slots as will be seen in some of the examples.

FIG. 11 shows a sleeve as in FIG. 6 that has been modified according to the present invention. In this instance, the slots have two differing geometries on each side of a segment. The slots 40 are open at the distal rim 15 and closed by stress relief holes at the proximal end of the slot. Each slot has jogs that create mating tabs between adjacent segments 32 and 32′ of the sleeve 10. Unlike a sleeve having a solid base 37 as in the previous examples shown in FIGS. 8, 9 and 10, the sleeve of FIG. 11 will not generate significant hoop stress at any region as long as the slots are not compressed closed. Also the surface pressure will be more consistent than the previous example because the effects of the rim and bending of the segments are eliminated. However, such a sleeve will not provide the sealing characteristics or provide the same type of taper fit created in the region of the base 37 of the previous examples.

FIGS. 11A, 11B and 11C show a close-up view of the region of the jog in the slot 40. Starting from the open bottom of the rim 15, the slot progresses approximately in a direction parallel to the axis C until it makes a series of 90 degree turns. The slot traverses leftward in a direction perpendicular to axis C than distally parallel to axis C than leftward and perpendicular to axis C and finally proximally parallel to axis C and concludes at the relief hole 38.

As shown in FIG. 11A, such a slot configuration creates two tabs, a first tab 42 projecting upward from a leftward segment 32 and a second tab 46 projecting downward from a rightward segment 32′. The gap also defines a pocket 44 that encloses the tab 42 and the pocket 48 that encloses the tab 46. In the neutral position shown in FIG. 11A, the gap is substantially equal between the tabs and between the body of segments 32 and 32′.

FIG. 11B shows the situation where the segments 32 and 32′ of the sleeve are expanded outward in a radial direction with respect to the axis C, for instance, by being fit on a prepared femoral head 7′. As the sleeve is installed, pressure at the bone sleeve interface forces the segments 32 and 32′ outward and apart to generally increase the gap, however, as this occurs, the tabs 42 and 46 move toward each other such that eventually they come in contact as shown. Features such as tabs 42 and 46 allow the sleeve to have a restraint on radial expansion which is not available in the straight slot configuration shown in FIGS. 8 and 9. This has the advantage, for instance, that if the preparation of a femoral head surface 9′ is un-symmetrical or the section of bone, as is often the case, is more rigid in a given portion of the prepared femoral head 7′, excessive radial deflection of a given segment will be limited once the tabs 42 and 46 engage.

It should be noted that unlike the situation in compression where the slot closes over a substantial length and the hoop stress is interrupted and distributed over a large area of the sleeve, any load from segment 32 to 32′ must travel through the tabs 42 and 46 and the deflection of the tabs 42 and 46. Thus the configuration of the tabs 42 and 46 can be varied to create a controlled rigidity. For example, if more rigidity is desired the base of each tab can be made longer and if the engaging surfaces of the tabs 42 and 46 are angled relative to each other the initiation of resistance from contact between the pads could start at a low level and progress and more of tab 42 is progressively engaged with tab 46.

FIG. 11C shows that in compression, segments 32 and 32′ will travel toward each other, closing the gap over most of the length of the slot 40 except that the tabs 42 and 46 move apart creating a larger gap 44.

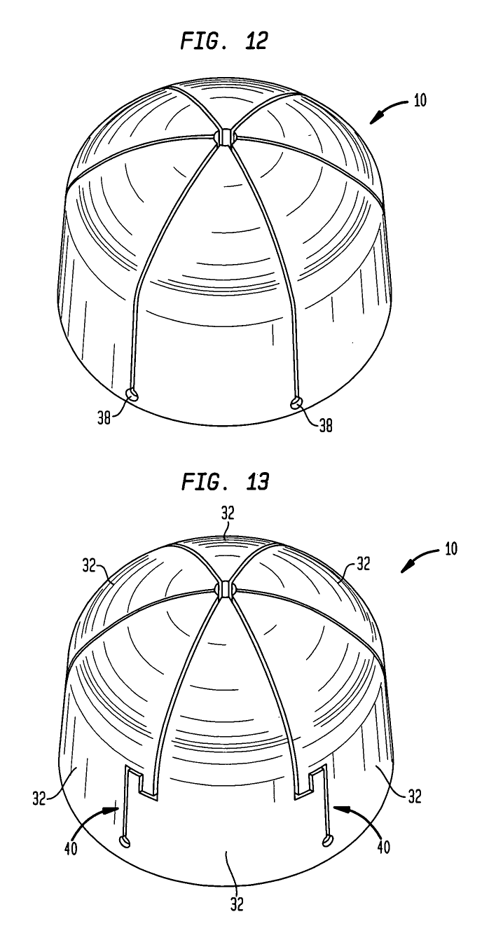

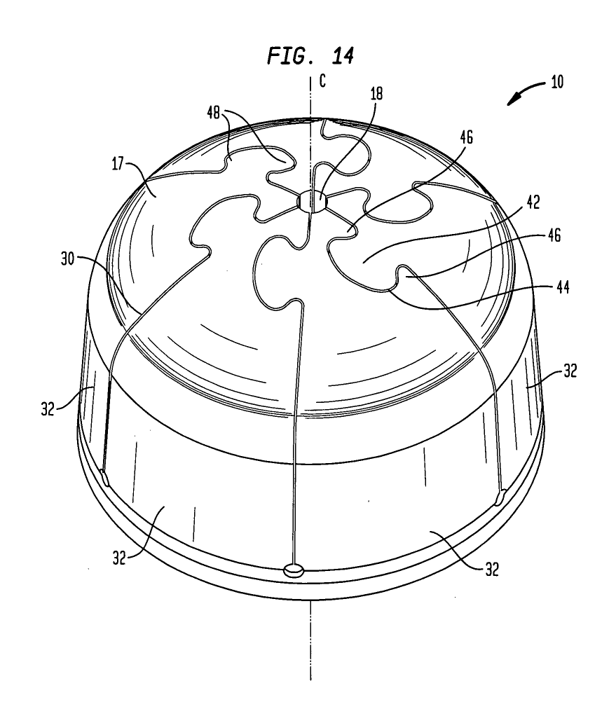

FIG. 12 shows a sleeve 10 as in FIG. 8 with a reduced rim 11 and without a relief groove 36. In this instance, the rim will still provide a solid sealed surface. The deflection can be characterized as a circumferential compression of the relief holes 38 in the area of the rim 11. FIG. 13 shows a combination of features of the sleeve 10 found in FIGS. 8, 9 and 10 with the jogging slot and tabs as shown in FIG. 11 to limit expansion of the segments 32.

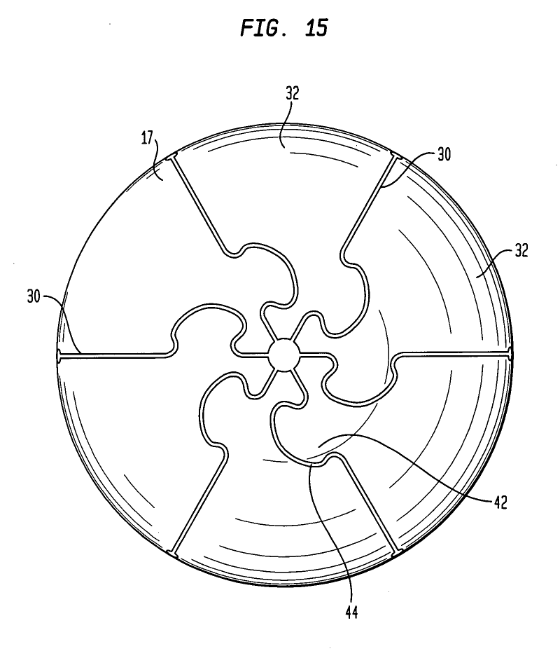

FIG. 14 shows a preferred embodiment of an aspect of the invention where features having some of the advantages shown in the embodiment of FIG. 11 are further advanced to limit radial expansion of the segments 32 in the proximal dome region 12 by a use of slots creating interlocking segments 32 not unlike the features of a jigsaw puzzle. Each slot 30 has, in the proximal dome section 12, a kidney shaped leftward jog and return before terminating in a central hole 18, creating a kidney shaped cavity 44. On the leftward side of each section 32, a tab 42, having projecting lobes 48, is engaged in cavity 44 by projecting extensions 46. The lobes and extensions restrain the relative movement of the tab 42 with respect to the cavity 44 to the width of the gap created by the slot 30 in all directions in the plane of the surface of the sleeve.

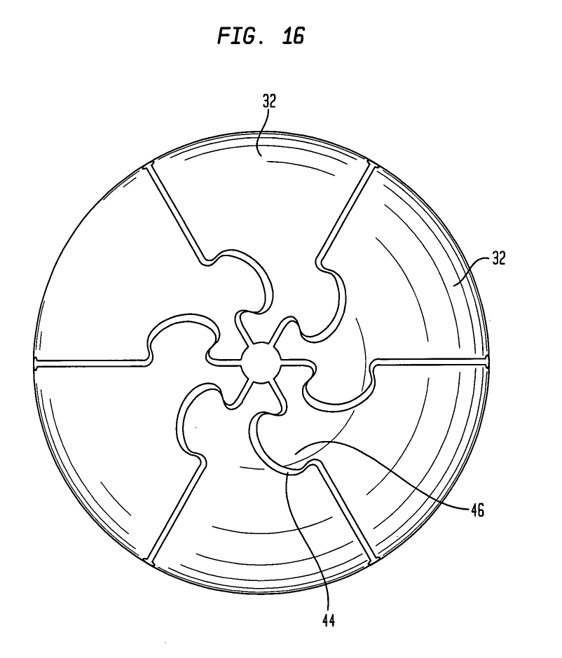

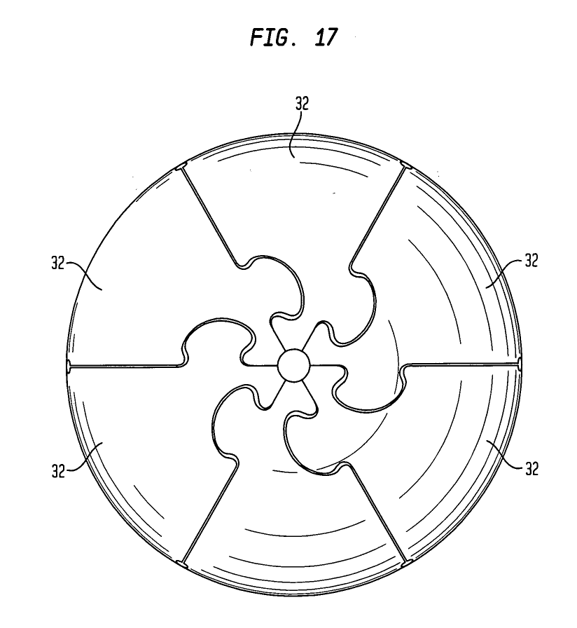

FIG. 15 is a plan view of the proximal surface 17 of the sleeve in FIG. 14 showing the arrangement of segments 32 with slots 30 in a neutral or free state. In this instance, the gaps created by the slots 30 are more or less equal over their extent including the regions between the tabs 42 and the cavities 44. FIG. 16 shows the same view where the segments 32 are radially expanded until the neck region defined by the extension 46 engages the tab 42 to limit travel. It should be noted that the shape of the lobes and cavities, etc. can be varied, as long as the corresponding features engage and interlock. By varying the geometries of the engaging regions, the stiffness and onset of stiffness of the restraint created by the engaged sections during the closing of the gaps can be adjusted as desired. FIG. 17 shows the same sleeve in a plan view under compression with the gaps closed up everywhere except in the regions of the extensions 46.

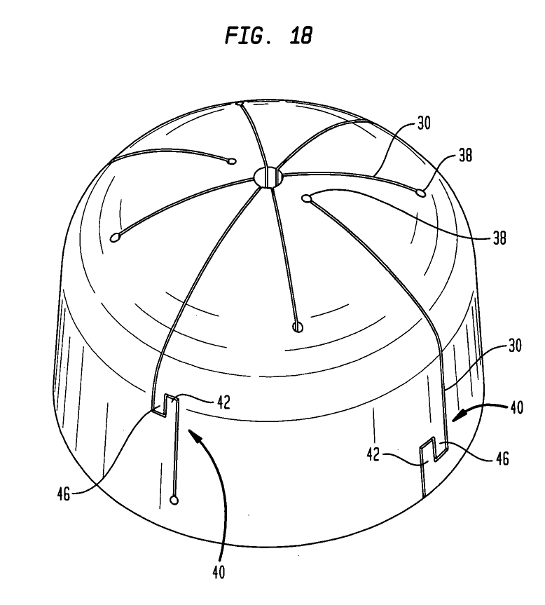

FIG. 18 shows a sleeve that is a hybrid of a sleeve with the features shown in FIGS. 8 and 9 and the features shown in FIG. 11. Thus, in this instance, the interlocking tabs shown in FIG. 11a are adapted to a sleeve 10 having a proximal dome portion. In this instance, the tab features 40 are at two different heights on the distal outer surface 15 and the slots are located only in the dome region to provide additional flexing in the dome. Thus, during extension of a given segment 32 in the dome region the travel of the segments apart is not limited by the features of the slots, but in the region of surface 15, radial travel of the segments will be limited at the various positions of the interlocking tabs.

In another aspect of the invention, it is desirable to vary the medial lateral position of the ball with respect to the proximal end of the prepared femoral head surface 9′ along the femoral neck axis B-B. This variation may be required when the surgeon, depending on the quality of the most proximal bone or the blood supply to the femoral remnant, needs to remove a significant part of the ephiphyseal bone or to adjust, for example, leg length.

Shown in FIGS. 1, 1A and 1B, the fused epiphyseal plate scar 8 created by the growth pattern of the femur 1 can be seen in the proximal portion of the prepared head 7′. It has been suggested by, for example, U.S. Pat. No. 4,662,888 that it is desirable to create the interface surface shaping proximate and preferably lateral to the fused epiphyseal plate scar 8 because the scar normally represents a natural division between arterial blood supplies in the bone and therefore excision of bone medial to the scar will leave the remaining lateral bone with blood supplied in a relatively normal manner to enhance the prospects of continued vitality of the resected bone.

In determining the extent of surgical preparation or resection with respect to the axial direction B-B for a given resection profile, the surgeon must balance the goal of bone preservation, the vitality of the existing bone and the ongoing vitality of the bone due to factors such as the location of the epiphyseal plate scar 8. Further, if the preparation position with respect to the axial direction B-B is to be varied for any reason, it is desirable that the implant may be adjustable to vary the position of the prosthetic femoral head along the axial direction B-B to establish an appropriate bio-mechanical joint geometry.

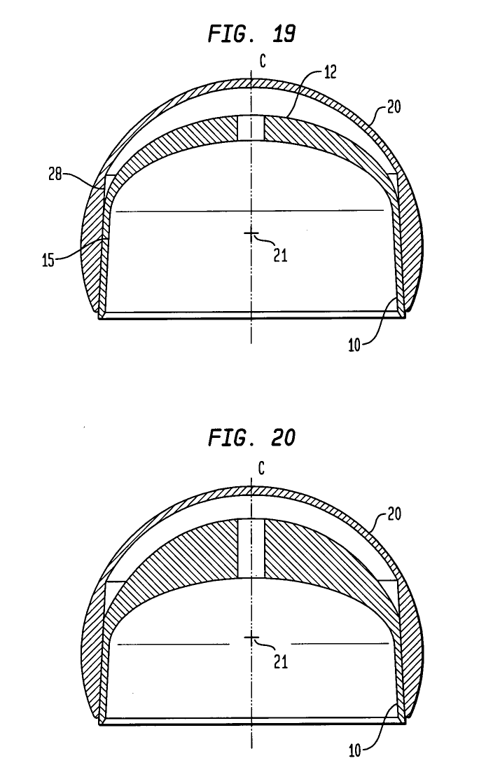

In an embodiment of the invention shown in FIGS. 19, 20 and 21, the center 21 of the ball component 20 is linearly offset along the axis C by varying the configuration of the sleeve 10. Typically the axis C is coincident with the femoral neck axis B-B and thus the ball center can be repositioned to allow the surgeon to vary the extent of the femoral head preparation or to otherwise adjust the position of the ball along the axis B-B to correct other concerns, such as leg length. Shifting of the position of the ball component 20 along the axis C is created by varying the relationship of the interface dimensions to create a translational offset. For example, in the instance of a conical interface, a relative increase of the R_{d} dimensions to the sleeve outer surface 15 with respect to the mating surface 28 of the ball will shift the ball component 20 in the proximal direction along axis C. Other dimensions, such as the thickness of the proximal dome region 12 are also suitably adjusted

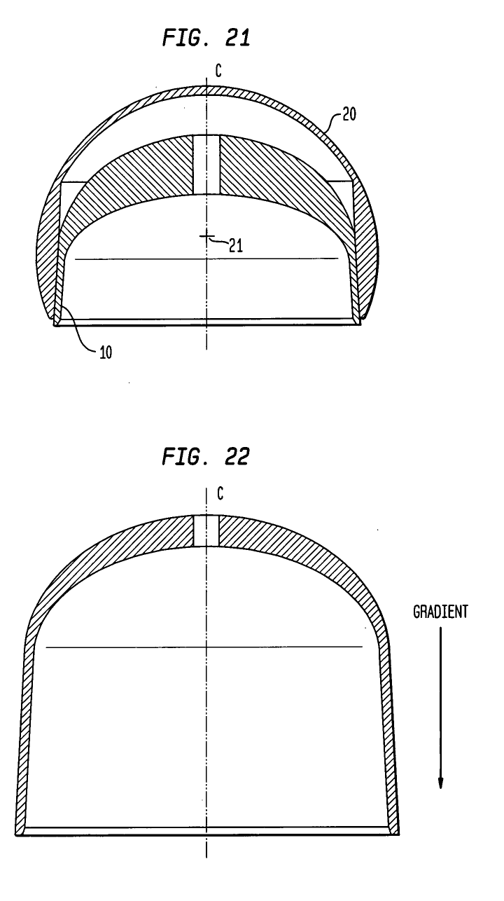

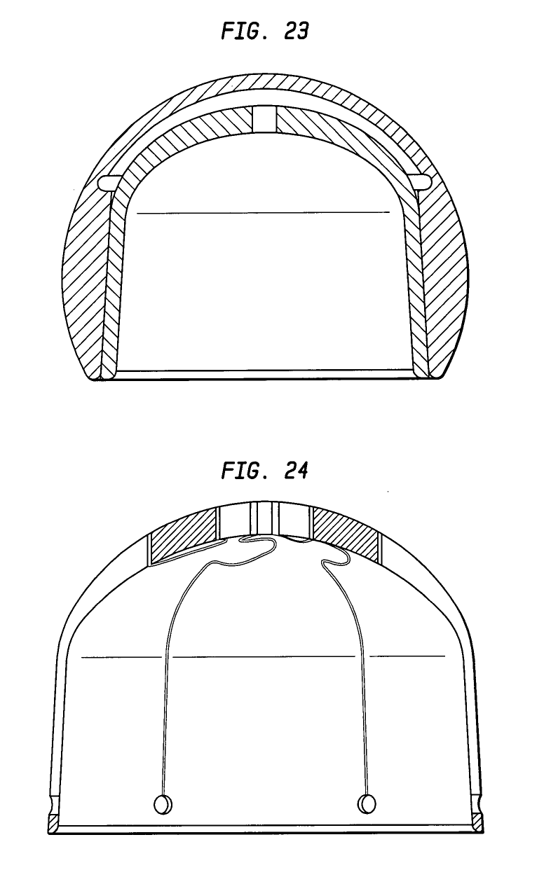

In another embodiment of the invention the load transfer from the prosthesis to the bone is optimized by creating a stiffness gradient between the bone and the head. To accomplish this, the stiffness of the sleeve is adjusted depending on the type of the bone it is interfacing with. Typically, the most proximal and superior bone is the stiffest bone while the bone facing the underside of the sleeve is softer. Thus a sleeve having a higher stiffness in the dome portion than in the bottom portion, as shown in FIGS. 22 and 23 helps to restore physiological loading of the bone and prevent relatively high stresses in the rim area of the sleeve. Importantly, a segmented sleeve is inherently less stiff and allows tailoring of the stiffness of the sleeve to an effective modulus that more closely matches the bone, yet has sufficient mechanical integrity to support and retain the ball component.

The gradient of stiffness can be achieved by variation of the thickness and porosity of the sleeve. The production method for such a sleeve can be by known methods of creating a gradient porosity as discussed above or using conventional manufacturing technology and drilling such as electron-beam, laser, electrical discharge machining.

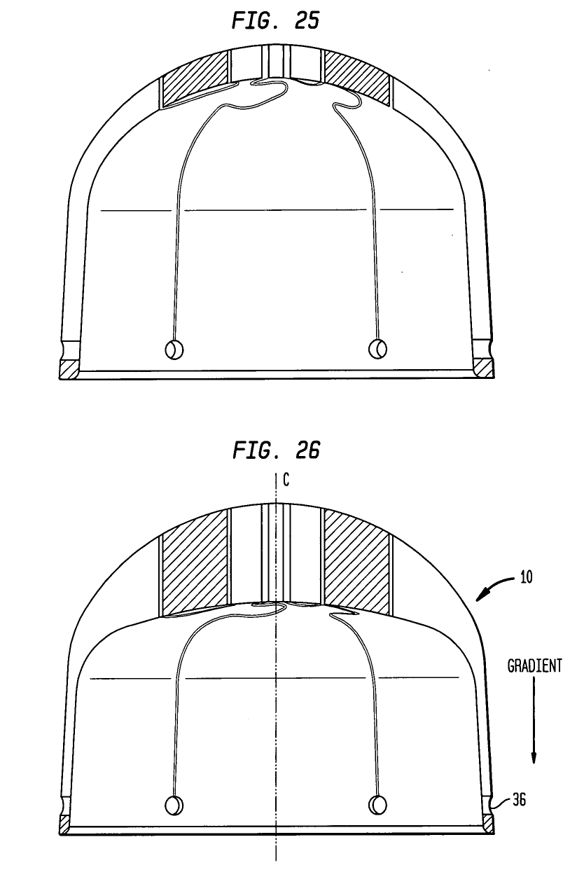

In another embodiment of the invention, variations of the sleeve lengths and thickness are used to adjust the prosthesis to the patient, particularly with respect to adjusting the head-neck ratio. With a modular construction having a head and a sleeve, it is possible to have various sleeve lengths and/or thicknesses in order to better fit the anatomy of the patient. For a patient having a rather small head-neck ratio, the sleeve can be thin and maximize the diameter of the mouth of the sleeve as shown in FIG. 24. For a patient having a large head-neck ratio, the sleeve may be thicker for the same head diameter as shown in FIG. 25. Therefore, for a given head diameter the surgeon would have the opportunity to prepare the femoral head in an optimum shape to preserve the patient-specific head-neck ratio by selecting an appropriate thickness sleeve.

FIG. 26 shows a variation of FIG. 24 that combines various aspects of the invention. A thin segmented sleeve according to the present invention is provided for a patient who has a relatively small head-neck ratio and requires the ball component to be moved proximally along the axis C-C. The sleeve 10 is segmented and has interlocking segments in the dome region. The proximal dome region 12 is thickened and therefore relatively stiffer to better match the modulus of the resected bone. Increased porosity in the distal tapered region and a groove 36 are used to further reduce the stiffness in the distal portion of the sleeve and better match the material properties of the distal portion of the resected femoral head 7′.

Thus it can be seen that the various aspects of the invention are synergistic and provide a comprehensive solution to the problems of the prior art when a porous or textured ingrowth surface is used for sleeve retention. Namely, fitting issues are solved because the sleeve can temporarily expand to a controlled additional clearance from the head, initial retention issues are solved because a press fit is created when the ball component is fitted, and bio-dynamic problems are solved because the sleeve allows correction of the ball component position on the sleeve, and adaptability to bone configurations and variations of bone physical characteristics.

A finite element study of a sleeve modeled after the sleeve of FIG. 14 confirms that the flexible sleeve 10 requires less force to install on the bone and develops a greater contact pressure with the bone, once the ball component is seated, than a rigid sleeve. For the configuration modeled, the contact pressure was approximately four times higher for an implant with a flexible sleeve compared with an implant with a rigid sleeve, given substantially the same material properties and installation forces. This modeling suggests that an implant with a flexible sleeve will provide greater implant stability along with easier installation.

As an optional variation of the invention, the gaps created by the cuts 30 may be filled or covered with a resilient gasket or seal (not shown) that still allows the segments 32 to flex substantially independently and effectively transmit force applied to the conical outer surface 15 and the proximal surface 12 of the sleeve 10 to the prepared femoral head surface 9′. Such a resilient gasket may be a material with a substantially lower modulus of elasticity than that of the segments 32, such as a polymer. The gasket may also take the form of a folded seal or bellows that expands and contracts to allow movement of the segments 32. If the parent material of the segments is suitably resilient, for example of a titanium alloy, the bellows may be formed integrally with the cuts 30.

The modular components of an implant according to the embodiments of the invention described above are particularly well suited for inclusion in a kit that can be used by a surgeon to evaluate and construct an implant specifically tailored to the patient's anatomy and dimensions. Such a kit of ball and sleeve components can include not only the usual variety of sizes of ball components etc. to fit the implant to the patient but also include sleeve components with altered geometries, segmentation and porosity gradients to facilitate variation in of the ball component position along the neck axis, and adaptation of resection geometries to different head-neck ratios.

The kit may also contain trial components, such as trial sleeve components that facilitate selection of the sleeve and ball components to actually be fitted to the patient by duplicating various aspects of the sleeve and ball components geometry. The trial components may include features that ease trial fitting but are not possible on an actual component. These features can include transparent components to allow visualization of otherwise obscured regions. External markings, orienting guides and tooling points can also be provided on the trial components. Features can also be incorporated to ease trial fitting, such as taper lock type features that provide accurate positioning, but do not readily lock or can be readily unlocked so as to more easily allow trial fitting of implant components.

Another aspect of the invention is to provide a method for installing the femoral sleeve prosthesis described above and, subsequently, a ball component by appropriately preparing and shaping the femoral head, guiding and seating the sleeve to a proper orientation on the prepared femoral head, and guiding and orienting the ball component onto the sleeve to complete the installation of the prosthesis. After the bone is prepared with the adequate instruments, the sleeve is driven onto the bone and slightly pushed (by hand or gently with a light mallet and sleeve driver). When it is pushed onto the bone, the cuts allow the sleeve to expand. The expansion is limited by the tabs 42 and 46. When the sleeve has stopped its expansion, the surgeon can check whether the sleeve has reached its final position. If it is not the case, it is possible to remove the sleeve, rework the bone and seat the sleeve again.

Once the sleeve is seated at its final position, the head is driven onto the sleeve. Because of the tapered connection (between 3° to 12°, preferably between 6° to 9°), the head is applying compression forces inwardly and provokes the compression of the bone/sleeve interface. The compression can theoretically happen as long as the cuts are not completely closed but will be limited by the resistance of the bone.

The various aspects of the kit described above may also be used during the surgical procedure. It will also be appreciated that even after fitting the actual ball component to the sleeve, the ball component can be removed and a ball component with a different offset or diameter can be used to alter the position of the bearing surface.

As an example of the method of installing a femoral prosthesis to a femoral ball or head, the outer surface of femoral head is first reamed and otherwise shaped to a predetermined configuration to match the shape of the sleeve and create a prepared femoral head having the desired head axis orientation; then a sleeve according to the embodiments of the invention discussed above is fitted on the prepared femoral head. If desired, the segments of the sleeve may be flexed outward by an installation tool acting on the segments, for example at the central hole 18 to hold the segments outward and further ease installation, especially if coarse textured features or spikes extend from the sleeve inner surface 14. A ball component according to the above is then fitted to the tapered sleeve surface and pressure is applied to lock the sleeve to the bone and the ball to the sleeve.

It will be appreciated that in a revision surgery or during the initial surgery, the original ball component can be removed and a new ball component can be fitted to the original sleeve to replace a ball component or to revise the position of the bearing surface. A new sleeve can also be fitted to, for example, adjust the position of the ball along the neck axis.

Unless stated to the contrary, any use of the words such as “including,” “containing,” “comprising,” “having” and the like, means “including without limitation” and shall not be construed to limit any general statement that it follows to the specific or similar items or matters immediately following it.

Although the invention herein has been described with reference to particular embodiments, it is to be understood that these embodiments are merely illustrative of the principles and applications of the present invention. It is therefore to be understood that numerous modifications may be made and are encouraged to be made to the illustrative embodiments and that other arrangements may be devised without departing from the spirit and scope of the present invention as defined by the appended claims.

1. A femoral prosthesis adapted to be installed to a prepared natural femoral head, the prepared natural femoral head having an outer surface and a head axis defined by symmetry with said outer surface, comprising:
a sleeve having
a) an open distal end and a proximal end;
b) a cavity bounded by an inner surface;
c) a conical outer surface at or adjacent said distal end, said conical outer surface having at least one gap dividing said sleeve into regions capable of separate radial deflection in response to radial force applied to said regions; and
a partial ball component having an open distal end, and a cavity bounded by a conical inner surface adapted to fit said conical outer surface of said sleeve.

2. The femoral prosthesis as set forth in claim 1 wherein at least a portion of said inner surface is a bone ingrowth surface.

3. The femoral prosthesis as set forth in claim 2 wherein said bone ingrowth surface comprises a porous surface.

4. The femoral prosthesis as set forth in claim 1 wherein interlocking tab surfaces formed by said at least one gap limits outward deflection of said regions.

5. The femoral prosthesis as set forth in claim 1 wherein said at least one gap is sized to close at a predetermined inward deflection of said regions.

6. The femoral prosthesis as set forth in claim 3 wherein said at least one gap is sized to close at a predetermined outward deflection of said regions.

7. The femoral prosthesis as set forth in claim 1 wherein said distal end of said outer conical surface is uninterrupted by said at least one gap.

8. The femoral prosthesis as set forth in claim 1 wherein said radial deflection is allowed by flexing said regions.

9. The femoral prosthesis as set forth in claim 1 wherein said proximal end of said sleeve comprises a dome.

10. The femoral prosthesis as set forth in claim 1 wherein said proximal end of said sleeve comprises a chamfered surface.

11. The femoral prosthesis as set forth in claim 1 wherein said inner surface of said sleeve comprises a conical inner surface.

12. The femoral prosthesis as set forth in claim 1 wherein said proximal end of said sleeve has a central aperture.

13. The femoral prosthesis as set forth in claim 1 wherein the outside of said sleeve is solid metal.

14. The femoral prosthesis as set forth in claim 3 wherein said porous bone ingrowth surface has a different porosity adjacent the proximal end than adjacent the distal end.

15. The femoral prosthesis as set forth in claim 1, wherein said sleeve is substantially composed of a metal selected from the group of titanium, titanium alloys, cobalt chrome alloys, niobium and tantalum.

16. The femoral prosthesis as set forth in claim 1, wherein said bone ingrowth surface is coated with a material selected from the group of bone morphogenic protein, calcium hydroxyapatite, tri-calcium phosphate, and antibiotics.

17. The femoral prosthesis as set forth in claim 1, wherein said at least one gap incorporates a resilient seal.

18. A kit for use in installing a femoral prosthesis on a prepared natural femoral head, the prepared head having an outer surface and a head axis defined by symmetry with said outer surface, said kit comprising:
A first sleeve having an open distal end and a proximal end, said sleeve having, an open distal end and a proximal end, a cavity bounded by an inner surface, and a conical outer surface at or adjacent said distal end, said conical outer surface having at least one gap dividing said sleeve into regions capable of separate radial deflection in response to radial force applied to said regions, said conical outer surface having a reference diameter d, said cavity having a geometry determining a coordinate system along said sleeve axis, said conical outer surface having a first translational position, determined by a reference diameter d, along said sleeve axis relative to said coordinate system; and
A second sleeve substantially similar to said first sleeve except said conical outer surface having a second translational position, determined by said reference diameter d, along said sleeve axis relative to said coordinate system.

19. The kit as set forth in claim 17 wherein said cavity has a porous inner surface.

20. A method of installing a femoral prosthesis to a femoral ball or head, the femoral head being coupled to the upper end of the main portion of the femur by a neck, said head and neck having a center and a central axis, said femoral head having an outer surface and an outer end, said method comprising the steps of:
a.) reaming the outer surface of the femoral head to a predetermined configuration to create a prepared femoral head having a head axis;
b.) fitting a sleeve to said prepared femoral head, said sleeve having, an open distal end and a proximal end, a cavity bounded by an inner surface, and a conical outer surface at or adjacent said distal end, said conical outer surface having at least one gap dividing said sleeve into regions capable of separate radial deflection in response to radial force applied to said regions; and
c.) fitting a partial ball component having an open distal end, and a cavity bounded by a conical inner surface adapted to fit said conical outer surface of said sleeve and apply radial force to said conical outer surface of said sleeve.

21. The method as set forth in claim 19 wherein said cavity has a porous inner surface.
